(19) United States
(12) Patent Application Publication
(10) Pub. No.: US 2022/0175987 A1
(43) Pub. Date: Jun. 9, 2022

(54) UVC-ENHANCED ELECTROMAGNETIC RADIATION WITH REFLECTIVE SHIELDS AND MATS

(71) Applicant: CUNNINGHAM, Emmett, Manuel, Glenn Allen, VA (US)

(72) Inventor: Emmett Manuel Cunningham, Glen Allen, VA (US)

(21) Appl. No.: 17/442,799

(22) PCT Filed: Mar. 25, 2020
(86) PCT No.: PCT/US2020/024606

§ 371 Date: Sep. 24, 2021

Related U.S. Application Data
(60) Provisional application No. 62/919,656, filed on Mar. 25, 2019.

(51) Int. Cl. A61L 2/10; H05B 47/115

(52) U.S. Cl. CPC A61L 2/10; H05B 47/115; A61L 2202/11; A61L 2209/15; A61L 2202/25; A61L 2209/111; A61L 2202/14

(57) ABSTRACT

Ultraviolet light, and in particular, UVC electromagnetic radiation hard surface and atmospheric disinfection system is able to totally disinfect a room or space by the sheer volume of UVC output and designed reflectance of the system. The reflectivity of the surface material of the device can be as high as 97% giving the UVC electromagnetic radiation, with a wavelength of 180 nm to 280 nm, almost twice the output looking forward. UVC radiation is not a straight path wavelength; the devices multi-faceted reflectance surfaces help overcome UVC's pathway divergence making its bacterial kill rate higher. The systems short-wave germicidal UVC light assemblies are both self-dependent and co-dependent; if you have a room with an adjoining bathroom you can place a single unit there with the same results.

### CROSS-REFERENCE TO RELATED APPLICATIONS

This application claims the benefit of co-pending U.S. Provisional Patent Application Ser. No. 62/919,656, filed 25 Mar. 2019, which is hereby incorporated herein as though fully set forth.

### FIELD OF THE INVENTION

The present invention relates to systems for the disinfection of hard-surfaces and atmospheric disinfection devices, more particularly, to UVC electromagnetic eradication of single cell life on hard surfaces and the surrounding air masses.

### BACKGROUND OF THE INVENTION

Disinfection of the hard surfaces and the surrounding air mass environment in today's hospitals is paramount to reducing hospital-acquired infections (HAI), which are a major challenge to patient safety. In American hospitals alone, the Centers for Disease Control (CDC) estimates that HAIs account for an estimated 1.7 million infections and 99,000 associated deaths each year.

There are many different approaches to the problem: fogging hydrogen peroxide, taking up to four hours (extremely dangerous and can destroy or bleach products in the room), fogging chlorine based products, taking up to one hour (leaves residue and can destroy or bleach products in the room), fogging ozone, taking up to two hours (extremely dangerous and hazardous to all mammals, requiring protective gear to operate), and ultraviolet C (UVC) irradiation, taking up to one hour. The machines come in every shape and size—stand alone, pulsing, flashing, mutable units (3) and, of course, robotic.

Focusing on the UVC devices, they all use tubes that emit in the range of 254 nm (e.g., 253.7 nm) and have an effective disinfection rate of 80-85% at one meter (about three feet). All of these devices are controlled by the limited amount of power in a hospital room, 20 amps, 110 VAC. This often limits the UVC tubes employed, which have a weaker output. The lower output and wattage also reduces their range, leaving their effective range below the industry standard of one meter. Except for systems that come with three standing units using the standard 254 nm tubes (but still limited to the 20 amps supplied), all of these devices have to be moved throughout the room in order to overcome the “shadow areas” (the areas that are not line of site within their one meter range).

### BRIEF DESCRIPTION OF THE DRAWINGS

These and other features of the invention will be more readily understood from the following detailed description of the various aspects of the invention taken in conjunction with the accompanying drawings that depict various embodiments of the invention, in which:

FIG. 1 shows a view of a disinfecting device according to an embodiment of the invention as it might be deployed within a hospital room;

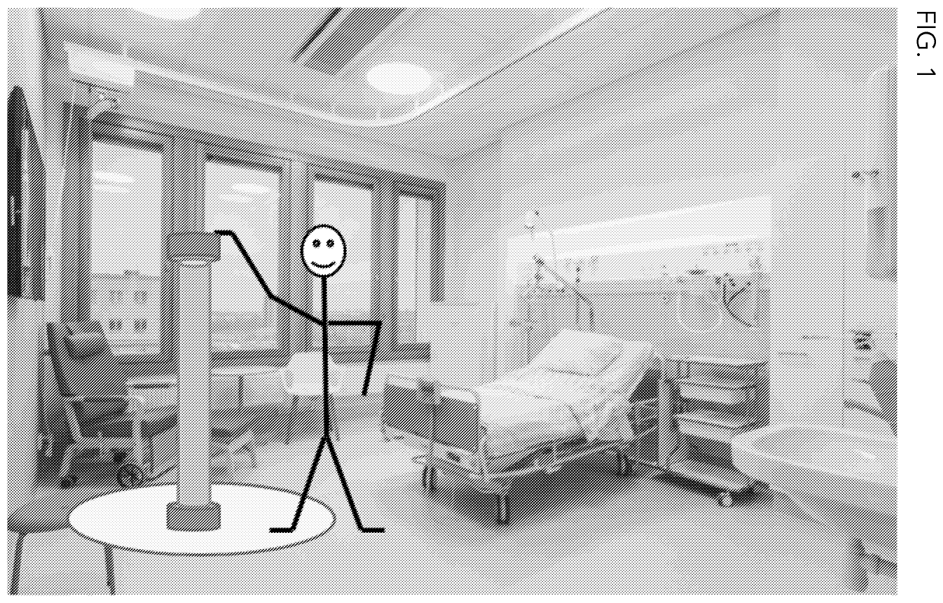

FIG. 2 shows a top schematic view of a system according to an embodiment of the invention, including a base unit and a plurality of satellite units, as may be deployed within a room, such as a hospital room;

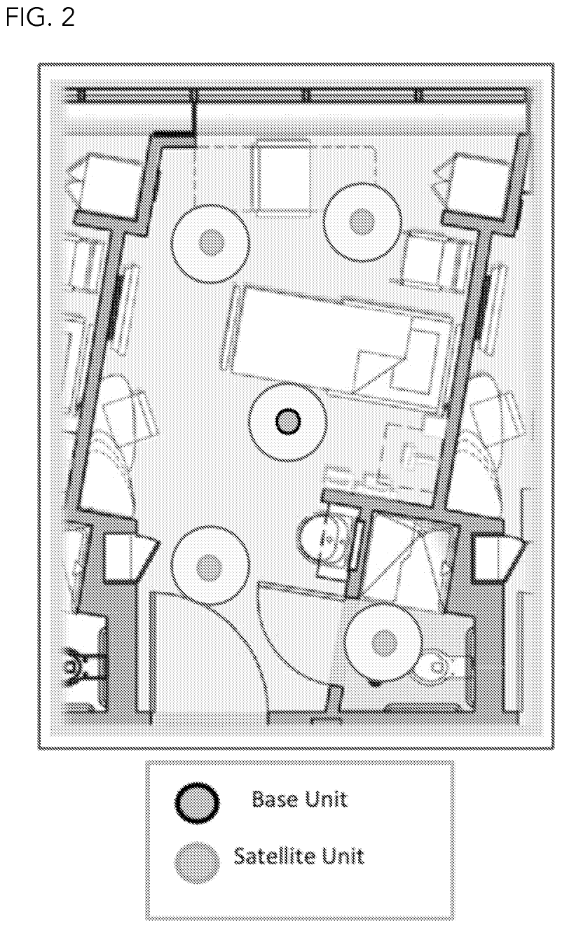

FIG. 3 shows graphical and side cross-sectional views of a disinfecting unit according to an embodiment of the invention;

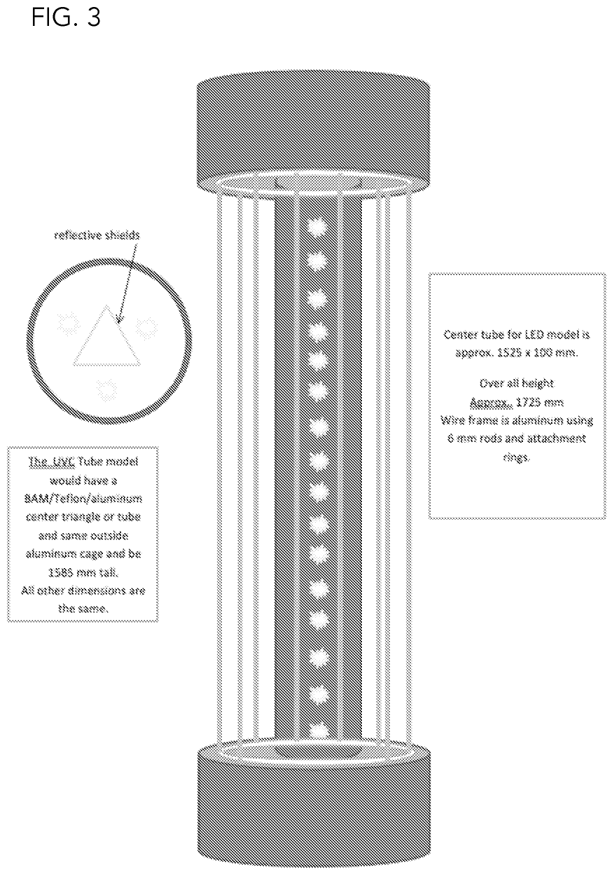

FIG. 4 shows top and bottom components and a cross-sectional view of a disinfecting unit according to another embodiment of the invention;

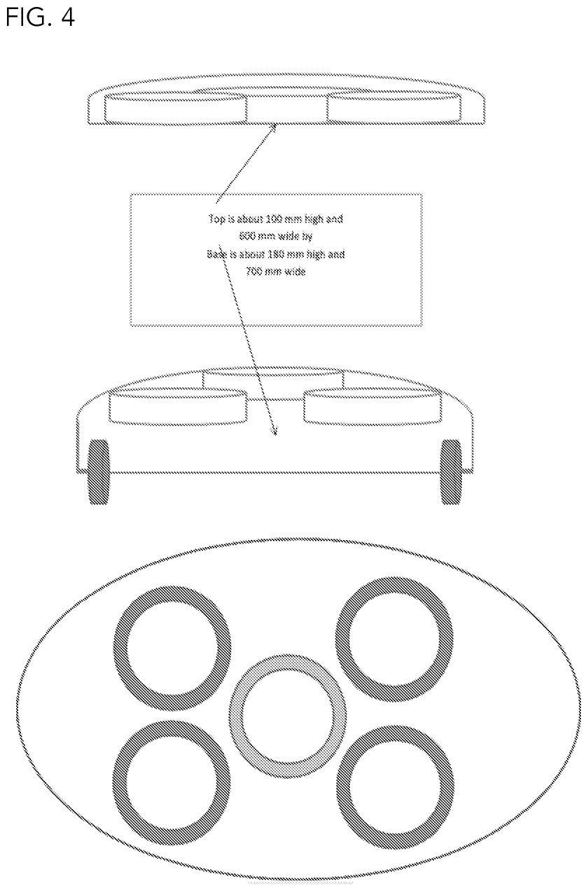

FIG. 5 shows a graphical perspective view of a disinfecting unit according to an embodiment of the invention that employs a reflective mat for reflecting upward UVC radiation; and

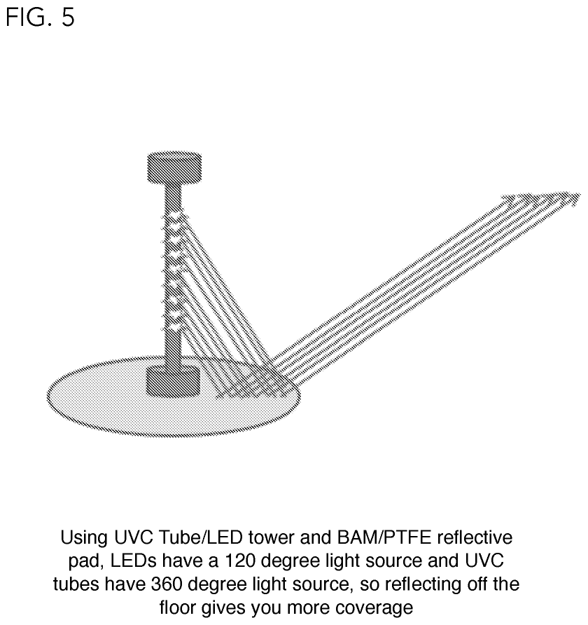

FIG. 6 shows a graphical top view of an arc of effective coverage of a disinfecting device according to an embodiment of the invention.

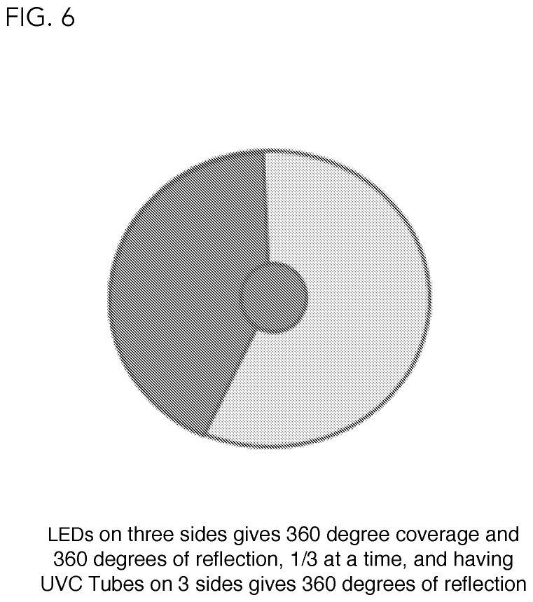

It is noted that the drawings are not to scale and are intended to depict only typical aspects of the invention, and therefore should not be considered as limiting the scope of the invention.

### DETAILED DESCRIPTION

For all the reasons above this invention was the result of needs—the need for safety of all personnel, the need to reduce the downtime of a critical patient's room (to under 10 minutes according to some embodiments of the invention), and most of all the need to disinfect the room by 92-97% using LEDs at 265 nm or UVC Tubes at 254 nm or 265 nm. One design has a “base station” with a plurality of satellites to be placed all around the room or space with one, if needed, to be placed in an adjoining room or space (e.g., a connected bathroom).

The invention is able to divide the supplied power into two separate 20 amp systems. The invention further reduces the power needs by reducing the number of UVC lamps (three per base or satellite), increasing their UVC output power (range) to 800 UV output at one m (μw/cm²) at 300 watts and almost doubling their output by the use an extremely low lubricity combined with very high reflectivity material that refocuses or reflects up to 97% of the electromagnetic UVC radiation to the area of disinfection.

By placing these “reflective shields” between the UVC bulbs or LEDs, the effective UVC radiation is reflected 160 degrees (80 degrees in both directions has shown to be the highest level of reflective UVC radiation, 97%). Suitable material options suitable for use in practicing the invention include: (1) boron, aluminum and magnesium (AlMgB₁₄) with titanium boride (TiB₂), BAM, with a static coefficient of friction of 0.02 and an electromagnetic radiation refection rating of 92-97%, (2) polytetrafluoroethylene (PTFE), having a static coefficient of friction of 0.04 and an electromagnetic radiation refection rating of 80-85%, (3) aluminum, having a static coefficient of friction of 0.47 and an electromagnetic radiation refection rating of 60-65%, and (4) stainless steel, having a static coefficient of friction of 0.5 and an electromagnetic radiation refection rating of 35-40%.

Adding to the reflective capabilities of this invention is a mat placed under each unit to reflect up to 97% of the unused UVC rays hitting the floor directly around each unit and redirecting them to the underside of furnishings in the enclosed room or space.

The base station and the mutable satellite units may include, for example, three stacks of UVC bulbs, each having a 160 degree active area, or a three- or four-sided stack of high output UVC LEDs with a 120 degree active area, giving the invention sufficient overlap of radiation of up to 480 degrees for full room/space coverage.

When working in the higher UVC range of 180 nm to 280 nm and the preferred range of 260-270 nm, a great deal of heat may be generated by the emitting devices. Because of the relatively short cycle time of this invention, any corresponding issues may be eliminated or alleviated with the addition of a heat sink, like aluminum, or a liquid circulation system. By making the light assembly tower, out of aluminum, one can connect the UVC output devices to it to reduce heat buildup in the system. Alternatively, the system may include cooling fins that carry a cooling liquid throughout the system tower.

To further the effectiveness of the system the “base station” and the base and top of the satellite units have a multi-faceted surface, of one of the high reflectivity materials from above, to reflect/deflect the electromagnetic UVC radiation in many directions, thereby eliminating all the “shadow areas” in the room or space.

The terminology used herein is for the purpose of describing particular embodiments only and is not intended to be limiting of the disclosure. As used herein, the singular forms “a,” “an,” and “the” are intended to include the plural forms as well, unless expressly states otherwise or the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described element, event, or circumstance may or may not occur, and that the description includes instances where the element, event, or circumstance occurs or is present and instances where it does not occur or is not present.

The corresponding structures, materials, acts, and equivalents of all means or step plus function elements in the claims are intended to include any structure, material, or act for performing a function in combination with other claimed elements as specifically claimed. The description of the present disclosure is presented for purposes of illustration and description, but is not intended to be exhaustive or limited to the disclosure in the form disclosed. Many modifications and variations will be apparent to those of ordinary skill in the art without departing from the scope and spirit of the disclosure. Any embodiments chosen and described herein appear in order to best explain the principles of the disclosure and their practical application, and to enable others of ordinary skill in the art to understand the disclosure for various embodiments with various modifications as are suited to the particular use contemplated.

1. A close-proximity short-wave germicidal ultraviolet C (UVC) light assembly disinfection system comprising:
at least one independently placeable germicidal electromagnetic UVC radiation assembly.

2. The system of claim 1, further comprising a base station.

3. The system of claim 2, wherein the at least one independently placeable germicidal electromagnetic UVC radiation assembly includes a plurality of independently placeable germicidal electromagnetic UVC radiation assemblies, each independently placeable about the base station.

4. The system of claim 1, wherein the at least one independently placeable germicidal electromagnetic UVC radiation assembly has an effective disinfection range of between about one meter and about six meters.

5. The system of claim 1, wherein the at least one independently placeable germicidal electromagnetic UVC radiation assembly includes a UVC light source having an emission range of 100 nm to 280 nm.

6. The system of claim 1, wherein the UVC light source includes a UVC light emitting diode (LED) with an emission range of 260 nm to 270 nm.

7. The system of claim 1, further comprising:
a motion detector.

8. The system of claim 1, further comprising:
at least one reflective shield comprising a material or having a coating with a low static coefficient of friction and a high reflectivity for electromagnetic radiation.

9. The system of claim 8, wherein the coating includes at least one compound selected from a group consisting of: AlMgB₁₄ (BAM) and TiB₂.

10. The system of claim 9, wherein the coating has a static coefficient of friction of about 0.02 and an electromagnetic radiation reflectivity rating between 92% and 97%.

11. The system of claim 8, wherein the coating includes polytetrafluoroethylene (PTFE).

12. The system of claim 11, wherein the coating has a static coefficient of friction of about 0.04 and an electromagnetic radiation reflectivity rating between 80 and 85%.

13. The system of claim 8, wherein the at least one reflective shield includes aluminum as a heat sink.

14-18. (canceled)

19. A method for disinfecting an enclosed space comprising:
placing one or more short-wave germicidal ultraviolet C (UVC) light assemblies in the enclosed space; and
activating said one or more short-wave germicidal UVC light assemblies to emit UVC radiation into the enclosed space.

20. (canceled)

21. (canceled)

22. The method of claim 19, wherein the one or more short-wave germicidal UVC light assemblies include a reflective shield comprising a material or having a coating with a low static coefficient of friction and a high reflectivity for electromagnetic radiation.

23-33. (canceled)

34. The method of claim 19, wherein the one or more short-wave germicidal UVC light assemblies is robotically controlled.

35. The system of claim 1, wherein the at least one independently placeable germicidal electromagnetic UVC radiation assembly is robotically controlled.

36. The system of claim 35, wherein the at least one independently placeable germicidal electromagnetic UVC radiation assembly is operable to produce UVC radiation in the range of 180 nm to 280 nm.

37. The system of claim 35, wherein the at least one independently placeable germicidal electromagnetic UVC radiation assembly is operable to produce UVC radiation in the range of 260 nm to 270 nm.
